(19) United States
(12) Patent Application Publication
(10) Pub. No.: US 2009/0131958 A1
(43) Pub. Date: May 21, 2009

(54) TWO-PART CLAMPING DEVICE

(75) Inventor: Bernard de Canniere, Brussels (BE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cardio Life Research S.A., Louvain-La-Neuve (BE)

(21) Appl. No.: 12/276,177

(22) Filed: Nov. 21, 2008

Related U.S. Application Data
(63) Continuation-in-part of application No. 10/836,712, filed on Apr. 29, 2004, now abandoned.
(60) Provisional application No. 60/467,810, filed on May 2, 2003.

(51) Int. Cl. A61B 17/08

(52) U.S. Cl. 606/158; 606/157

(57) ABSTRACT

A surgical clamping device for hollow anatomical structures such as, essentially, blood vessels, but also tracheas, intestines, etc. This device comprises three initially independent parts, namely two jaws, each jaw being formed of an elongate member having distal and proximal ends, and a mandrel that can be slipped over the proximal ends of the two jaws and that is able to bring the axes of the two jaws closer together, thus pinching an anatomical structure. The mandrel comprises a distal, pinching part and a proximal, handling part, these two parts being joined together by a locking part, the jaws being bendable at the level of the locking part when the clamp is activated, the distal, pinching part of the mandrel remaining in place by friction on the jaws when it is unlocked from the proximal, handling part. This surgical clamping device applies more particularly to the clamping of the aorta.

### CROSS-REFERENCES TO RELATED APPLICATIONS

The present application is a continuation-in-part of U.S. patent application Ser. No. 10/836,712, filed Apr. 29, 2004, which claims the benefit of U.S. Provisional Patent Application Ser. No. 60/467,810, filed May 2, 2003. The entire disclosures of both applications are incorporated by reference in their entirety.

### BACKGROUND OF THE INVENTION

The invention relates to clamping devices for hollow tubular anatomical structures such as, essentially blood vessels, but also for tracheas, intestines, etc.

### TECHNICAL BACKGROUND OF THE INVENTION

Heart surgery usually requires stopping the heart so as to obtain a stationary and exsanguinous operating site allowing a precise and delicate surgical operation.

Sternotomy is a destructive surgical approach that carries significant post-operative risks to the patient.

Furthermore, clamping the aorta is an operation which is considered to be delicate and high risk because, in particular, of the proximity of the pulmonary artery, the texture of which is known to be extremely fragile.

What is more, clipping the artery using a conventional clamp is a source of embolism of atheromatous material which, in most cases, lines the internal wall of the vessel.

For many years, heart surgery has been developing alternative techniques aimed at being less aggressive toward the patient. Doing away with the sternotomy is one of these approaches. In this case, the operation is carried out using mini-incisions allowing endoscopic instruments to be introduced.

An alternative solution allowing a great amount of patients to undergo minimally invasive heart surgery in complete safety was developed by the same inventor and disclosed in PCT/BE01/00211.

In PCT/BE01/00211 was disclosed a surgical clamp which comprised a flexible guide; two jaws each formed of an elongate member pierced with a longitudinal canal and having a distal end and a proximal end. Said jaws were so designed that they could be slipped over the guide with their proximal end facing toward each of the ends of the guide; a mandrel that can be slipped over the two ends of the guide and over the jaws and which is able to bring the axes of the two jaws closer together and thus perform pinching.

The technique developed in PCT/BE01 00211 now begins to be applied by numerous surgeons. However, the practice soon proved that slight changes in the design could improve dramatically the efficiency of the basic clamp.

In micro-surgery, owing to the restricted space the surgeon has to deal with, it is obvious that the operating field should be cleared out as quickly as possible of non-essential instruments. The clamp plays an essential role and has to be put in place at the very beginning of the operation and remains in place for a long time. The problem was to render it as little cumbersome as possible.

### BRIEF SUMMARY OF THE INVENTION

The non-obvious solution that was found was to manufacture the clamp in separate parts, so that only an active, pinching head thereof remains in place, its proximal, handle part being removed from the way.

The advantage of the clamp of the invention is that the clamping remains effective and powerful through a minimal incision.

Another problem that arises in micro-surgery is the fact that the incision through which the clamp is to be inserted is of very restricted diameter, and further close to the organs to be clamped, so that there remains little room to manipulate the jaws, particularly if they have to be inserted at different angles.

Accordingly, the clamp of the invention is manufactured so that its different parts (namely, two jaws and a mandrel) are separate and fully independent of each other, so that each jaw can be inserted in full independency of the other jaw, the two jaws being interlocked merely upon insertion of the mandrel along their proximal parts.

The device of the invention also applies particularly advantageously to the clamping of the aorta by using the anatomical space of Theile's transverse sinus as a natural guide, but it further can be used without guides in operations wherein the structure that has to be clamped is easy to reach.

Another advantage is that the risk of damaging an adjacent organ is reduced to a minimum, the disturbing proximal part being pulled aside and interfering thus no more with other instruments used by the surgeon.

The clamp of the invention can be used with equal ease for intra-thoracic and extra-thoracic vascular structures and for other anatomical structures including, in particular, the intestines. It may also be used as forceps for manipulating bones.

In another aspect of the present invention, a method for clamping a hollow organ of a patient is provided. The method includes providing a clamp composed of three basic elements that are initially completely separate from each other: a first elongated rod, where a distal end of the rod forms a jaw, a second elongated rod, where a distal end of the rod forms another jaw, and a mandrel. The mandrel is adapted to be slipped over the proximal ends of the first and second elongated rods when they are placed side-by-side. The mandrel comprises a distal, pinching part and a proximal, handling part, where these two parts are joined together by a locking part. A mini-incision is made in the body of the patient in the vicinity of the organ to be clamped. The distal end of the first elongated rod of the clamp is inserted through the mini-incision and positioned along a first side of the organ to be clamped. Next, the distal end of the second elongated jaw of said clamp is inserted through the mini-incision and positioned along a second side of the organ to be clamped, where the second side is opposite the first side of the organ to be clamped. The proximal ends of the first and the second elongated rods are lined up and inserted into a distal end of the mandrel. The mandrel is slid towards the distal end of the rods, thus bringing the distal ends of the first and the second elongated jaws closer to each other and pinching the organ to be clamped. The locking means of the mandrel are unlocked and the distal, pinching part of the mandrel is unlocked from the proximal, handling part, such that the distal, pinching part of the mandrel remains with the first and second elongated jaws, together forming a distal part of the clamp. The distal part of the clamp is moved aside to allow the surgeon access to the surgical site to perform an operation. At the end of the operation, the proximal, handling part of the mandrel is slid along the proximal part of the elongated rods until it reaches the distal, pinching part of the mandrel. The distal, pinching part is then re-locked with the proximal, handling part of the mandrel and the mandrel is removed, thus unclamping the organ. The jaws are then removed from the body of the patient.

### BRIEF DESCRIPTION OF THE DRAWINGS

Other particulars and advantages of the invention will become apparent from the description hereinafter of some particular embodiments of the invention, reference being made to the appended drawings in which:

FIG. 1 is a perspective schematic view of the clamping of a human heart in the case of an operation with a sternotomy.

FIG. 2 is a view with cutaway of a heart operation with a mini-intercostal incision.

FIG. 3 is a view of one form of an embodiment of the clamp of the invention prior to its insertion, and thus in unassembled form.

FIG. 4 is view in perspective of the clamp of FIG. 3 in active, pinching position,

FIG. 5 is view in perspective of the clamp of FIG. 3 with unlocked mandrel parts.

FIG. 6 is a view in section of a pair of jaw elements of the clamp of the invention.

FIGS. 7a and 7b are views of clamping operations using prior art devices.

FIGS. 7c and 7d are views of a clamping operation with the clamp of the invention.

FIG. 8 is a perspective view another embodiment of the locking means of the mandrel.

### DETAILED DESCRIPTION OF THE INVENTION

FIG. 1 shows the various operations in preparation for a conventional heart operation, so as to obtain an exsanguinous and stationary operation field.

Venous blood (low in oxygen) is diverted down a cannula 2 as it enters the heart 4 via the right atrium 6 toward a heart-lung machine (not depicted) which namely reoxygenates it and rids it of its CO₂. The artificially oxygenated blood is then returned by a second cannula 8 at the aorta 10 into the patient's arterial circuit, thus short-circuiting the heart 4 and the pulmonary circulation so as to allow the intra-cardiac or extra-cardiac operation to be performed.

The heart 4 can therefore be stopped in order to obtain an exsanguinous and stationary operating field.

The heart is conventionally stopped using two joint operations:
clamping the aorta;
injecting a cardioplegy solution into the coronary circulation.

Clamping the aorta 10 consists in blocking the vessel using external forceps 12 which are applied between the arterial cannula 8 of the extra-corporeal circulation and the orifice of the coronary arteries 14. This operation isolates the coronary circulation from the blood flow generated by the ECC.

A cardioplegy solution can then be injected by an injection member 16 into the coronary circulation to “paralyze” the heart 4 with a view to allowing the surgeon to operate more precisely then he could on moving anatomical structures.

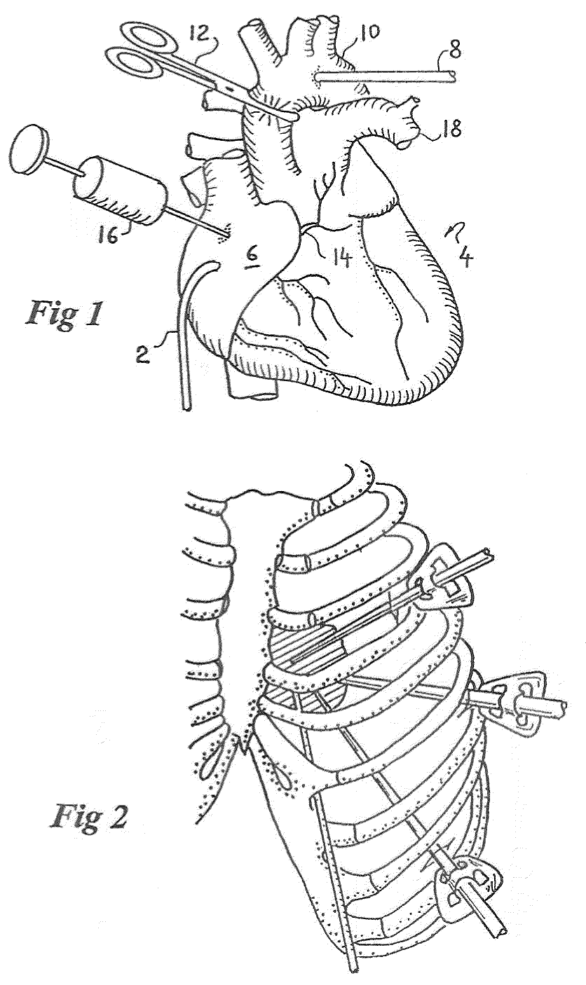

FIG. 2 shows another known approach, in which a heart operation is conducted via one or more incisions of the order of one centimeter long, allowing endoscopic instruments to be introduced.

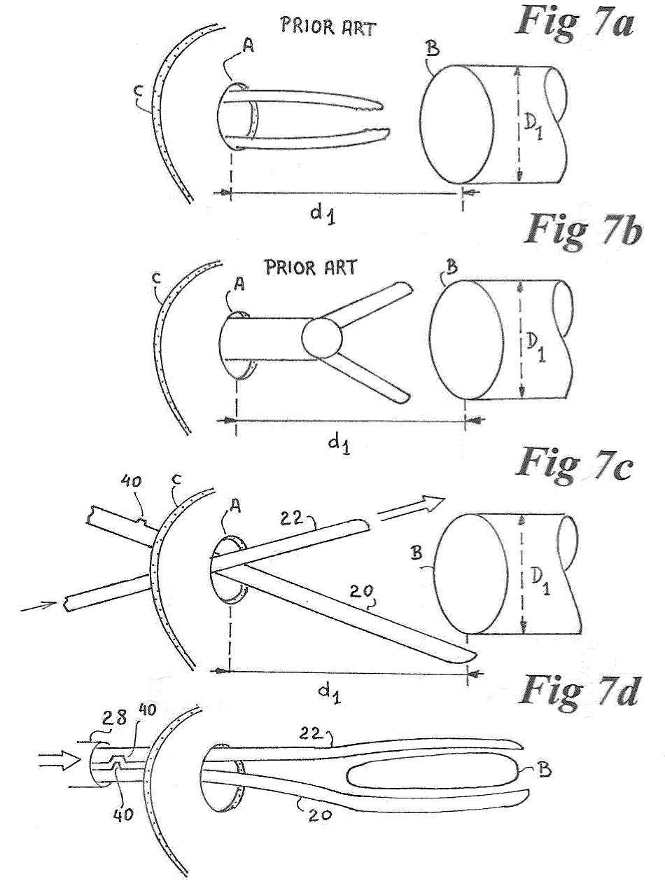

As was stated earlier, it is impossible to resort to conventional forceps in which the size of the jaws and their travel are out of proportion with the size of the intercostal incisions made (see FIGS. 7a and 7b).

The major advantage of the system according to the invention is that it allows the clamping to be performed without opening up the thorax but, what is more, with a lower risk of trauma to the pulmonary artery and of embolism.

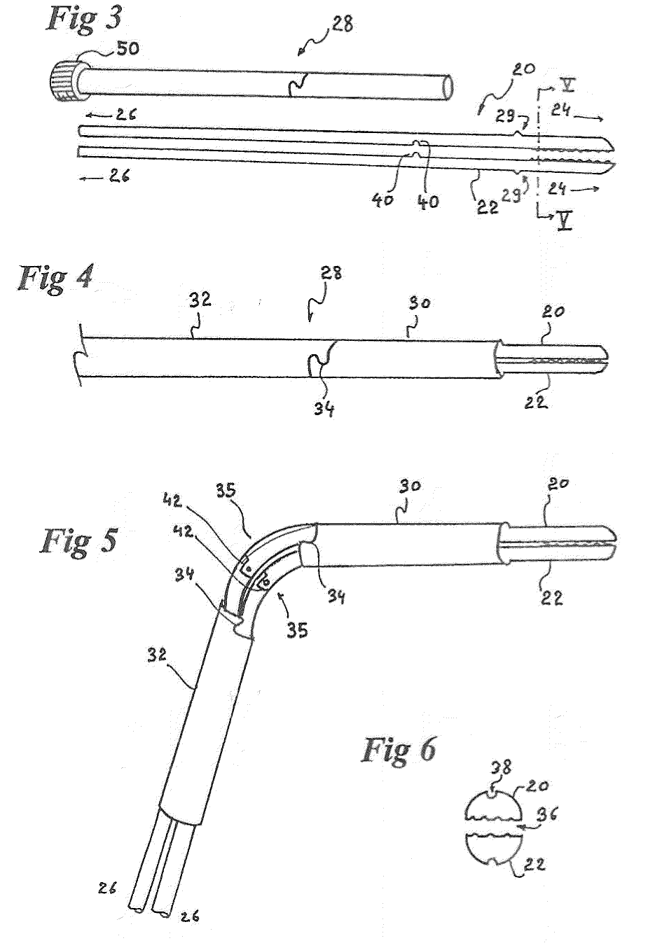

The clamping device of the invention and its various components will be described with reference to the succession of FIGS. 3 to 5.

Two semi-rigid bendable jaws 20, 22, each formed of an elongated rod having a distal, pinching end 24 and a proximal, handling end 26 are inserted in place carefully one after the other so that their distal ends can be positioned either side of a vessel, organ or structure to be clamped. The proximal ends of the jaws are then manually joined and their respective positions are rectified. A hollow mandrel 28 is then inserted around the proximal ends of both jaws 20, 22 and made to slide along the jaws toward their distal ends. The distal ends of the jaws 20, 22 then each align with the axis of the mandrel 28 and move closer together, causing gentle and gradual pinching of the vessel, as can be seen in FIG. 4. Abutment means 29 placed on the jaws 20, 22 limit the movement of the mandrel, so as not to bruise the vessel.

By “semi-rigid, bendable” jaws, it is meant that the rods may be bent outside the body to clear the access to the incision in the body, but rigid enough to effectively clamp shut the treated vessel, organ or structure. As seen in FIG. 6, the jaws are plain rods with a planar section. In the text hereafter, the term “rod” will be used interchangeably with the term “jaw,” as these two words describe the same element.

As described in PCT/BE01/00211, when the vessel is not easy to reach, the jaws and the mandrel may be slipped over a guide inserted beforehand around the vessel.

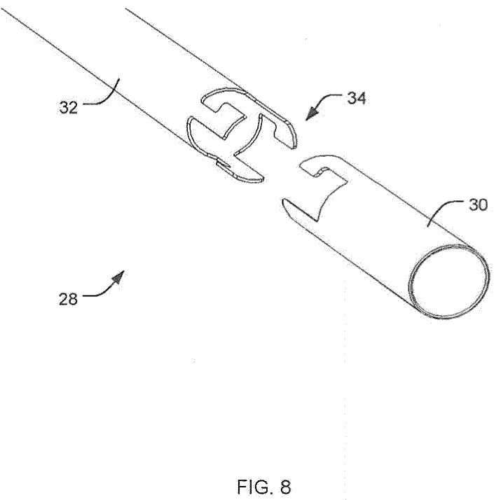

One main innovative part of the present invention is that the mandrel 28 itself comprises two parts (see, e.g., FIG. 8): a distal part 30 and a proximal, handling part 32, both parts being locked together by a locking fitting 34, here in the form of a bayonet, but which may also be a screw or other fastening.

When the organ or structure is firmly held between the jaws, the operator unlocks the locking fitting 34, here by simple twisting. The proximal, handling part 32 of the mandrel 28 may then be slightly pulled back (see FIG. 5) and the jaws 20, 22 comprising a flexible section 35 at the level or upstream of the locking fitting 34, the proximal part of the whole device can be pulled aside, disencumbering the operating field for the surgeon. The figures do not show to scale the various parts of the clamp. The full length of the rods 20, 22 (see FIG. 3) is of about 350 to 400 mm. The pinching, distal part thereof; that constitute properly the “jaws” protrude of about 60 mm from the distal part of the mandrel 30. The distal part of the mandrel has a length range between 20 and 40 mm, while the handling part 32 has a length of about 150-200 mm.

At the end of the intervention, the two parts of the mandrel 28 are lined up, re-locked together and the mandrel 28 is pulled back as a whole, causing the jaws to relax around the clamped structure, organ or vessel.

FIG. 6 shows, in cross section, the distal part of the jaws 20, 22. In this embodiment, these may be provided with flexible jaw elements or with ribbings so as to spread the pressure over the organ which has been grasped. This section also displays a longitudinal groove 38 allowing a guide to be passed along the jaws.

Each member forming a jaw 20, 22 is made of plastic. The faces of these jaws that face toward each other and are slightly toothed form the jaw elements 36. The back of each jaw 20, 22, comprises a longitudinal groove 38 intended to accommodate a flexible guide.

Each of the jaws carries, at its central part, alignment means 40 which are intended to engage in the corresponding parts of the other jaw so as to prevent any relative lengthwise movement of the jaws once their proximal ends have been interlocked by the insertion of the mandrel 28.

According to a form of embodiment, the mandrel 28 can be formed of a metal part bent over on itself.

To further improve the ease of use of the clamp, the jaws themselves may be provided with an unlocking feature 42, allowing a complete separation of the head part and the handling part, thus further disencumbering the operating field. As can be seen at FIG. 5, the unlocking feature can be, e.g., a combination of button and buttonhole or similar.

FIGS. 7a, 7b and 7c, 7d explain how the problem of cumbersomeness is solved in micro-surgery by a clamp of the invention. A is a mini-incision in the skin C, having a length on the order of 8 to 12 mm, through which a clamp has to be introduced in the body of a patient. B is an artery to be clamped, and D1 defines its diameter, which ranges between 10 and 50 mm, typically about 30 mm. The distance d1 between the mini-incision and the artery is on the order of 40 to 80 mm, typically about 60 mm, so that, as shown in FIG. 7a, the jaws of a conventional clamp such as described in U.S. Pat. No. 5,222,973 cannot grasp easily the whole artery. The solution in such a situation is either to enlarge the mini-incision A, or to use shorter jaws, such as those described in U.S. Pat. No. 6,162,239, (shown at FIG. 7b), but again the clamp then will not be able to grasp the whole artery B properly. Further, while trying to insert one of the jaws in place, the surgeon cannot ignore the presence of the second jaw, which is mechanically linked to the first jaw and which risks bruising or even piercing artery B or any other near organ, because the second jaw necessarily moves as the first jaw is positioned as a result of their linkage. In contrast, the jaws 20, 22 of the clamp of the present invention being fully independent, they can be inserted one by one according to distinct insertion angles, as shown at FIG. 7c, so that their distal end can easily be positioned on either sides of artery B. The proximal ends of the jaws are then lined up (see FIG. 7d) so that the mandrel 28 can be slid around their proximal ends, as explained above, and pushed forwards, thus gently clamping the artery B. Another advantage to be noted is that the present clamp is particularly easy to handle and to manipulate, the sole “manipulating part” remaining toward its proximal side when the clamp is in place being a knob-shaped handle 50 placed at the proximal end of the mandrel 28. The surgeon can, through this handle, apply a push, a pull or a torque to the mandrel.

While the invention has been described by way of example and in terms of the specific embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A surgical clamp comprising three separate elements initially completely separated from each other:
two separate semi-rigid jaws, each jaw being formed of an elongated rod extending along an axis and having a distal end and a proximal end, each jaw being adapted to be inserted in full independency on either side of an organ to be clamped;
a mandrel having a distal side and a proximal side, the distal side of which is adapted to slide from the proximal end on over the proximal ends of the jaws placed side-by-side and which is able to bring the distal end of the axes of the two jaws closer together after the jaws have been positioned on either side of the organ to be clamped, said mandrel comprising a distal, pinching part and a proximal, handling part, these two parts being joined together by locking means, the distal, pinching part of the mandrel remaining in place by friction on the jaws when it is unlocked from the proximal, handling part, the proximal, handling part being removable by sliding it off the proximal ends of the rods, the jaws being bendable at the level or upstream of the locking part when the clamp is assembled and locked.

2. A surgical clamp according to claim 1 wherein each jaw comprises a longitudinal canal for inserting a flexible guide.

3. A surgical clamp according to claim 1 having a slightly cylindrical section when assembled.

4. A surgical clamp according to claim 2 having a slightly cylindrical section when assembled.

5. The surgical clamp as claimed in claim 1, wherein the jaws have a malleable section at their proximal end.

6. The surgical clamp as claimed in claim 1, wherein the mandrel comprises a single longitudinal canal, the distal end of this canal being shaped in such a way as to cause the axes of the two jaws to be moved closer together by relative displacement.

7. The surgical clamp as claimed in claim 1, wherein the jaws are provided with flexible jaw elements near their distal end.

8. The surgical clamp as claimed in claim 1, wherein the jaws comprise abutment means for limiting the longitudinal movement of the mandrel.

9. The surgical clamp as claimed in claim 1, wherein the proximal part of the jaws is equipped with ribbing collaborating with a mechanism supported by the mandrel.

10. The surgical clamp as claimed in claim 9, wherein the proximal part of the jaws is equipped with ribbing collaborating with a mechanism supported by the mandrel.

11. The surgical clamp as claimed in claim 1, wherein the jaws further comprise a distal, pinching part and a proximal, handling part, these two parts being joined together by unlocking means.

12. The surgical clamp as claimed in claim 2, wherein the jaws further comprise a distal, pinching part and a proximal, handling part, these two parts being joined together by unlocking means.

13. The surgical clamp as claimed in claim 1, wherein the jaws further comprise alignment means preventing any relative lengthwise movement of the jaws once their proximal ends have been interlocked by the insertion of the mandrel.

14. A method for clamping a hollow organ of a patient, the method comprising:
providing a clamp comprising three basic elements, initially completely separated from each other: a first elongated rod, a distal end of said rod forming a jaw, a second elongated rod, a distal end of said rod forming a jaw, and a mandrel, wherein said mandrel is adapted to be slipped over proximal ends of the first and second elongated rods placed side-by-side, the mandrel further comprising a distal, pinching part and a proximal, handling part, these two parts being joined together by a locking part;
making a mini-incision in the body of the patient, in the vicinity of the organ to be clamped;
inserting through said mini-incision the distal end of the first elongated rod of the clamp;
positioning the distal end of said first elongated rod forming a jaw along a first side of the organ to be clamped;
inserting through said mini-incision the distal end of the second elongated rod of said clamp;
positioning the distal end of said second jaw along a second side of the organ to be clamped, said second side being at the opposite of the first side of the organ to be clamped;
lining up the proximal ends of the first and the second elongated rods;
inserting the proximal ends of the first and the second elongated rods into a distal end of the mandrel;
sliding the mandrel towards the distal end of the rods, thus bringing the distal ends of the first and the second elongated jaws closer to each other and pinching the organ to be clamped;
unlocking the locking means of the mandrel;
separating the distal, pinching part from the proximal, handling part of the mandrel, such that the distal, pinching part of the mandrel together with the first and second elongated jaws form a distal part of the clamp;
moving the distal part of the clamp aside to clear access to the mini-incision;
performing an operation on the patient;
at the end of the operation, sliding the proximal, handling part of the mandrel along the proximal part of the rods until it reaches the distal, pinching part thereof;
re-locking the distal, pinching part of the mandrel with the proximal, handling part of the mandrel;
removing the mandrel, thus unclamping the organ; and
removing the jaws from the body of the patient.
